(19) United States
(12) Patent Application Publication
(10) Pub. No.: US 2022/0192955 A1
(43) Pub. Date: Jun. 23, 2022

(54) AZOXYSTROBIN EFFICACY IN SCALP HEALTH

(71) Applicant: The Procter & Gamble Company, Cincinnati, OH (US)

(72) Inventors: Jeanette Anthea Richards, Liberty Township, OH (US); Kathleen Marie Kerr, Okeana, OH (US); Angela Marie Fieno, Hamilton, OH (US)

(21) Appl. No.: 17/126,975

(22) Filed: Dec. 18, 2020

(51) Int. Cl. A61K 8/49; A61Q 5/00; A61Q 5/02; A61Q 5/12

(52) U.S. Cl. CPC A61K 8/494; A61Q 5/006; A61K 2800/596; A61Q 5/12; A61K 2800/5426; A61Q 5/02

(57) ABSTRACT

The present invention is directed to use of azoxystrobin to decrease the number of flakes on a surface; the use of azoxystrobin to decrease itch on a surface as perceived by a user; the use of azoxystrobin to decrease the level of myeloperoxidase on a surface as an indicator of decrease of oxidative stress and damage. The present invention is directed to the use of azoxystrobin wherein the azoxystrobin is applied from a personal care composition such as a shampoo, conditioner or leave on treatment.

### FIELD OF THE INVENTION

The present invention is directed to azoxystrobin providing an improvement in scalp health including reduction of flaking on the scalp, reduction in scalp inflammation as well as reduction in perception of scalp itch. The present invention is further directed to use of azoxystrobin to provide scalp barrier improvement.

### BACKGROUND OF THE INVENTION

Dandruff and seborrheic dermatitis are conditions of the human scalp and skin that involve Malassezia yeasts as culprit organisms for initiation and/or exacerbation of unhealthy scalp/skin symptoms. Topical antifungals are routinely used for development of consumer products that address these conditions and discovery of antifungals that more effectively control the growth and effects of Malassezia on the scalp or skin is the focus of much research. Strobilurins represent one such class of antifungal agents used in agriculture for crop protection that are largely unexplored for growth control of Malassezia and its related skin diseases.

The present invention has found that azoxystrobin, a specific synthetic strobilurin, is unique among other compounds of this class in its antifungal potency against Malassezia. Evaluation of azoxystrobin on the scalp of consumers with dandruff, results in resolution of flaking and improvement of other scalp health symptoms. Topical antifungal drugs are often combined with anti-inflammatory drugs such as corticosteroids for the most effective management of symptoms such as inflammation and itch in severe disease states. It has been discovered that azoxystrobin has an unexpected effectiveness in improving overall scalp health on its own when used to treat the human scalp subject to the dandruff condition. These scalp health benefits involve reduction in inflammation and itch perception, which are benefits that cannot be predicted from its antifungal efficacy alone.

### SUMMARY OF THE INVENTION

The present invention is directed to use of azoxystrobin to decrease the number of flakes on a surface; the use of azoxystrobin to decrease itch on a surface as perceived by a user; the use of azoxystrobin to decrease the level of myeloperoxidase on a surface as an indicator of decrease of oxidative stress and damage. The present invention is directed to the use of azoxystrobin wherein the azoxystrobin is applied from a personal care composition such as a shampoo, conditioner or leave on treatment.

### DETAILED DESCRIPTION OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Leave-on,” in reference to compositions, means compositions intended to be applied to and allowed to remain on the keratinous tissue. These leave-on compositions are to be distinguished from compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on compositions” may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography. “QS” means sufficient quantity for 100%.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

### Azoxystrobin and Other Strobilurins

Azoxystrobin, CAS number: 131860-33-8, IUPAC: methyl-(E)-(2-{2[6-(2-cyanophenoxy)-pyrimidin-4-iloxy]-phenyl}-3-methoxyacrylate is an agricultural fungicide belonging to the class of the strobilurins. Strobilurins are either biosynthesized by various Basidiomycete fungi such as Strobilurus tenacellus and Oudemansiella mucida or modeled after natural strobilurins and synthesized with retention of the key β-methoxyacrylate toxophore. Some synthesized strobilurins have a modified toxophore e.g. methyl methoxyiminoacetate or methyl-N-methoxycarbamate. Some synthetic strobilurins are azoxystrobin (CAS number: 131860-33-8), coumoxystrobin (CAS number 850881-70-8), dimoxystrobin (CAS number 149961-52-4), enoxastrobin (CAS number 238410-11-2), fluoxastrobin (CAS number 193740-76-0), kresoxim methyl (CAS number 143390-89-0), mandestrobin (CAS number 173662-97-0), metominostrobin (CAS number 133408-50-1), orysastrobin (CAS number 248593-16-0), picoxystrobin (CAS number 117428-22-5), pyraclostrobin (CAS number 175013-18-0), pyraoxystrobin (CAS number 862588-11-2), and trifloxystrobin (CAS number 141517-21-7).

Azoxystrobin and other synthetic strobilurins control a broad spectrum of plant fungal disease and are used heavily in crop protection worldwide. Strobilurins work by inhibition of mitochondrial respiration. The specific mode of action of azoxystrobin and other strobilurins is by binding the ubiquinol oxidizing site (Q₀ site) in the cytochrome b complex III of the electron transport chain and blocking electron transfer between cytochrome b and cytochrome c₁. Other compounds with this specific mode of action include synthetic and naturally occurring derivatives of the key β-methoxyacrylate toxophore known as oudemansins also first isolated from Oudemansiella mucida, synthetic and naturally occurring myxothiazols from myxobacteria such as Myxococcus flavus, stigmatellins from myxobacteria such as Stigmatella aurantica and the synthetic agricultural chemicals famoxadone and fenamidone.

Azoxystrobin as an agricultural fungicide has protectant, curative, eradicant, translaminar and systemic properties and inhibits spore germination and mycelial growth, and also shows antisporulant activity. At labelled application rates, azoxystrobin controls the numerous plant pathogens including Erysiphe graminis, Puccinia spp., Lepiosphaeria nodorum, Septoria tritici and Pyrenophora teres on temperate cereals; Pyricularia oryzae and Rhizoctonia solani on rice; Plasmopara viticola and Uncinula necator on vines; Sphaerotheca fuliginea and Pseudoperonospora cubensis on cucurbitaceae; Phytophthora infestans and Alternaria solani on potato and tomato; Mycosphaerella arachidis, Rhizoctonia solani and Sclerotium rolfsii on peanut; Monilinia spp, and Cladosporium carpophilum on peach; Pythium spp. and Rhizoctonia solani on turf; Mycosphaerella spp. on banana; Cladosporium caryigenum on pecan; Elsinoe fawcetii, Colletotrichum spp. and Guignardia citricarpa on citrus; Colletotrichum spp. and Hemileia vastatrix on coffee. Azoxystrobin is a solid material having low solubility in water.

Some tradenames for azoxystrobin include ABOUND FLOWABLE FUNGICIDE, Aframe, Azoxystar, Azoxyzone, AZteroid 1.65 SC Fungicide, AZURE AGRICULTURAL FUNGICIDE, Endow, QUADRIS FLOWABLE FUNGICIDE, Satori Fungicide, Strobe 2L, and Willowood Azoxy 2SC. Azoxystrobin is commercially available from for example Sigma-Aldrich (St. Louis, Mo.) and Ak Scientific, Inc (Union City, Calif.).

In the present invention, the personal care composition may contain from about 0.02% to about 10% of azoxystrobin; from about 0.05% to about 2% of azoxystrobin; from about 0.1% to about 1% of azoxystrobin.

In the present invention, the personal care composition may contain from about 0.02% to about 10% of a strobilurin; from about 0.05% to about 2% of a strobilurin; from about 0.1% to about 1% of a strobilurin.

In the present invention, the particle size of azoxystrobin may be from about 0.5 microns to about 200 microns; from about 0.5 microns to about 100 microns; from about 1 micron to about 50 micron; from about 1 microns to about 25 microns, from about 1 microns to about 10 microns from about 1 micron to about 3 microns.

### Shampoo Compositions

### Detersive Surfactant

The personal care composition may comprise greater than about 10% by weight of a surfactant system which provides cleaning performance to the composition, and may be greater than 12% by weight of a surfactant system which provides cleaning performance to the composition. The surfactant system comprises an anionic surfactant and/or a combination of anionic surfactants and/or a combination of anionic surfactants and co-surfactants selected from the group consisting of amphoteric, zwitterionic, nonionic and mixtures thereof. Various examples and descriptions of detersive surfactants are set forth in U.S. Pat. No. 8,440,605; U.S. Patent Application Publication No. 2009/155383; and U.S. Patent Application Publication No. 2009/0221463, which are incorporated herein by reference in their entirety.

The personal care composition may comprise from about 10% to about 25%, from about 10% to about 18%, from about 10% to about 14%, from about 10% to about 12%, from about 11% to about 20%, from about 12% to about 20%, and/or from about 12% to about 18% by weight of one or more surfactants.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the personal care composition include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate or sodium laureth sulfate.

The composition of the present invention can also include anionic surfactants selected from the group consisting of:
a) R₁ O(CH₂CHR₃O)_{y} SO₃M;
b) CH₃ (CH₂)_{z} CHR₂ CH₂ O (CH₂ CHR₃O)_{y} SO₃M; and
c) mixtures thereof,
where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols, F-T oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfate and sodium trideceth-3 sulfate. The composition of the present invention can also include sodium tridecyl sulfate.

The composition of the present invention can also include anionic alkyl and alkyl ether sulfosuccinates and/or dialkyl and dialkyl ether sulfosuccinates and mixtures thereof. The dialkyl and dialkyl ether sulfosuccinates may be a C6-15 linear or branched dialkyl or dialkyl ether sulfosuccinate. The alkyl moieties may be symmetrical (i.e., the same alkyl moieties) or asymmetrical (i.e., different alkyl moieties). Nonlimiting examples include: disodium lauryl sulfosuccinate, disodium laureth sulfosuccinate, sodium bistridecyl sulfosuccinate, sodium dioctyl sulfosuccinate, sodium dihexyl sulfosuccinate, sodium dicyclohexyl sulfosuccinate, sodium diamyl sulfosuccinate, sodium diisobutyl sulfosuccinate, linear bis(tridecyl) sulfosuccinate and mixtures thereof.

The personal care composition may comprise a co-surfactant. The co-surfactant can be selected from the group consisting of amphoteric surfactant, zwitterionic surfactant, non-ionic surfactant and mixtures thereof. The co-surfactant can include, but is not limited to, lauramidopropyl betaine, cocoamidopropyl betaine, lauryl hydroxysultaine, sodium lauroamphoacetate, disodium cocoamphodiacetate, cocamide monoethanolamide and mixtures thereof.

The personal care composition may further comprise from about 0.25% to about 15%, from about 1% to about 14%, from about 2% to about 13% by weight of one or more amphoteric, zwitterionic, nonionic co-surfactants, or a mixture thereof.

Suitable amphoteric or zwitterionic surfactants for use in the personal care composition herein include those which are known for use in shampoo or other personal care cleansing. Non limiting examples of suitable zwitterionic or amphoteric surfactants are described in U.S. Pat. Nos. 5,104,646 and 5,106,609, which are incorporated herein by reference in their entirety.

Amphoteric co-surfactants suitable for use in the composition include those surfactants described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, thoseselected from the group consisting of: sodium cocaminopropionate, sodium cocaminodipropionate, sodium cocoamphoacetate, sodium cocoamphodiacetate, sodium cocoamphohydroxypropylsulfonate, sodium cocoamphopropionate, sodium cornamphopropionate, sodium lauraminopropionate, sodium lauroamphoacetate, sodium lauroamphodiacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphodiacetate, ammonium cocoamphohydroxypropylsulfonate, ammonium cocoamphopropionate, ammonium cornamphopropionate, ammonium lauraminopropionate, ammonium lauroamphoacetate, ammonium lauroamphodiacetate, ammonium lauroamphohydroxypropylsulfonate, ammonium lauroamphopropionate, ammonium cornamphopropionate, ammonium lauriminodipropionate, triethanolamine cocaminopropionate, triethanolamine cocaminodipropionate, triethanolamine cocoamphoacetate, triethanolamine cocoamphohydroxypropylsulfonate, triethanolamine cocoamphopropionate, triethanolamine cornamphopropionate, triethanolamine lauraminopropionate, triethanolamine lauroamphoacetate, triethanolamine lauroamphohydroxypropylsulfonate, triethanolamine lauroamphopropionate, triethanolamine cornamphopropionate, triethanolamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof

The composition may comprises a zwitterionic co-surfactant, wherein the zwitterionic surfactant is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof.

Suitable nonionic surfactants for use in the present invention include those described in McCutcheion's Detergents and Emulsifiers, North American edition (1986), Allured Publishing Corp., and McCutcheion's Functional Materials, North American edition (1992). Suitable nonionic surfactants for use in the personal care compositions of the present invention include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

The co-surfactant can be a non-ionic surfactant selected from the alkanolamides group including: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, PPG-2 Hydroxyethyl Isostearamide and mixtures thereof.

Representative polyoxyethylenated alcohols include alkyl chains ranging in the C9-C16 range and having from about 1 to about 110 alkoxy groups including, but not limited to, laureth-3, laureth-23, ceteth-10, steareth-10, steareth-100, beheneth-10, and commercially available from Shell Chemicals, Houston, Tex. under the trade names Neodol® 91, Neodol® 23, Neodol® 25, Neodol® 45, Neodol® 135, Neodo®1 67, Neodol® PC 100, Neodol® PC 200, Neodol® PC 600, and mixtures thereof.

Also available commercially are the polyoxyethylene fatty ethers available commercially under the Brij® trade name from Uniqema, Wilmington, Del., including, but not limited to, Brij® 30, Brij® 35, Brij® 52, Brij® 56, Brij® 58, Brij® 72, Brij® 76, Brij® 78, Brij® 93, Brij® 97, Brij® 98, Brij® 721 and mixtures thereof.

Suitable alkyl glycosides and alkyl polyglucosides can be represented by the formula (S)n-O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, lauryl alcohol, myristyl alcohol, cetyl alcohol, stearyl alcohol, oleyl alcohol, and the like. Examples of these surfactants include alkyl polyglucosides wherein S is a glucose moiety, R is a C8-20 alkyl group, and n is an integer of from about 1 to about 9. Commercially available examples of these surfactants include decyl polyglucoside and lauryl polyglucoside available under trade names APG® 325 CS, APG® 600 CS and APG® 625 CS) from Cognis, Ambler, Pa. Also useful herein are sucrose ester surfactants such as sucrose cocoate and sucrose laurate and alkyl polyglucosides available under trade names Triton™ BG-10 and Triton™ CG-110 from The Dow Chemical Company, Houston, Tex.

Other nonionic surfactants suitable for use in the present invention are glyceryl esters and polyglyceryl esters, including but not limited to, glyceryl monoesters, glyceryl monoesters of C12-22 saturated, unsaturated and branched chain fatty acids such as glyceryl oleate, glyceryl monostearate, glyceryl monopalmitate, glyceryl monobehenate, and mixtures thereof, and polyglyceryl esters of C12-22 saturated, unsaturated and branched chain fatty acids, such as polyglyceryl-4 isostearate, polyglyceryl-3 oleate, polyglyceryl-2-sesquioleate, triglyceryl diisostearate, diglyceryl monooleate, tetraglyceryl monooleate, and mixtures thereof.

Also useful herein as nonionic surfactants are sorbitan esters. Sorbitan esters of C12-22 saturated, unsaturated, and branched chain fatty acids are useful herein. These sorbitan esters usually comprise mixtures of mono-, di-, tri-, etc. esters. Representative examples of suitable sorbitan esters include sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), and sorbitan isostearate.

Also suitable for use herein are alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Also suitable for use herein are alkylphenol ethoxylates including, but not limited to, nonylphenol ethoxylates (Tergitol™ NP-4, NP-6, NP-7, NP-8, NP-9, NP-10, NP-11, NP-12, NP-13, NP-15, NP-30, NP-40, NP-50, NP-55, NP-70 available from The Dow Chemical Company, Houston, Tex.) and octylphenol ethoxylates (Triton™ X-15, X-35, X-45, X-114, X-100, X-102, X-165, X-305, X-405, X-705 available from The Dow Chemical Company, Houston, Tex.).

Also suitable for use herein are tertiary alkylamine oxides including lauramine oxide and cocamine oxide.

Non limiting examples of other anionic, zwitterionic, amphoteric, and non-ionic additional surfactants suitable for use in the personal care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

Suitable surfactant combinations comprise an average weight % of alkyl branching of from about 0.5% to about 30%, alternatively from about 1% to about 25%, alternatively from about 2% to about 20%. The surfactant combination can have a cumulative average weight % of C8 to C12 alkyl chain lengths of from about 7.5% to about 25%, alternatively from about 10% to about 22.5%, alternatively from about 10% to about 20%. The surfactant combination can have an average C8-C12/C13-C18 alkyl chain ratio from about 3 to about 200, alternatively from about 25 to about 175.5, alternatively from about 50 to about 150, alternatively from about 75 to about 125.

Deposition Aids

The shampoo compositions of the present invention may further comprise a deposition aid, such as a cationic polymer or cationic deposition polymer. Cationic polymers useful herein are those having an average molecular weight of at least about 5,000, alternatively from about 10,000 to about 10 million, and alternatively from about 100,000 to about 2 million.

The cationic polymer may be, including but not limited to a cationic guar polymer, has a weight average Molecular weight of less than 2.2 million g/mol, or from about 150 thousand to about 2.2 million g/mol, or from about 200 thousand to about 2.2 million g/mol, or from about 300 thousand to about 1.2 million g/mol, or from about 750,000 thousand to about 1 million g/mol. The cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.8 meq/g.

The cationic guar polymer may have a weight average Molecular weight of less than about 1.5 million g/mol, and has a charge density of from about 0.1 meq/g to about 2.5 meq/g. The cationic guar polymer may have a weight average molecular weight of less than 900 thousand g/mol, or from about 150 thousand to about 800 thousand g/mol, or from about 200 thousand to about 700 thousand g/mol, or from about 300 thousand to about 700 thousand g/mol, or from about 400 thousand to about 600 thousand g/mol or from about 150 thousand to about 800 thousand g/mol, or from about 200 thousand to about 700 thousand g/mol, or from about 300 thousand to about 700 thousand g/mol, or from about 400 thousand to about 600 thousand g/mol. The cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.5 meq/g.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums. A nonlimiting example of a cationic polymer is guar hydroxypropyltrimonium chloride.

The cationic polymer can be included in the hair care compositions of the present invention at a level of from about 0.001 wt. % to about 10 wt. %. In the present invention, the cationic polymer may be present in an amount up to about 5 wt % based on the weight of the composition.

### Aqueous Carrier

The personal care composition comprises an aqueous carrier. Accordingly, the formulations of the personal care composition can be in the form of pourable liquids (under ambient conditions). Such compositions will therefore typically comprise an aqueous carrier, which is present at a level of from about 20 wt. % to about 95 wt. %, or from about 60 wt. % to about 85 wt. %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carriers useful in the personal care composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, dipropylene glycol, hexylene glycol, glycerin, and propane diol.

### Emulsifiers

In cases where the personal care composition does not include a gel matrix, the 1,2-diol can be pre-emulsified before it is added in the personal care composition. Emulsifiers selection for each conditioning active is guided by the Hydrophilic-Lipophilic-Balance value (HLB value) of emulsifiers. Suitable range of HLB value is 6-16, more preferably 8-14. Emulsifiers with an HLB higher than 10 are water soluble. Emulsifiers with low HLB are lipid soluble. To obtain suitable HLB value, a mixture of two or more emulsifiers may be used. Suitable emulsifiers include non-ionic, cationic, anionic and amphoteric emulsifiers.

Rheology Modifier/Thickener

The personal care compositions mentioned above may also contain one or more rheology modifier/thickener to adjust the rheological characteristics of the composition for better feel, in-use properties and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. Further, the leave-on treatment may comprise from about 0.01% to about 3% of a rheology modifier, alternatively from about 0.1% to about 1% of a rheology modifier,

The one or more rheology modifier may be selected from the group consisting of polyacrylamide thickeners, cationically modified polysaccharides, associative thickeners, and mixtures thereof. Associative thickeners include a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. These materials may have a hydrophobe that can be selected from cetyl, stearyl, oleayl, and combinations thereof, and a hydrophilic portion of repeating ethylene oxide groups with repeat units from 10-300, alternatively from 30-200, and alternatively from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of additional rheology modifiers include acrylamide/ammonium acrylate copolymer (and)polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and/or combinations thereof.

A non exclusive list of suitable thickeners for use herein include xanthan, guar, hydroxypropyl guar, scleroglucan, methyl cellulose, ethyl cellulose (commercially available as Aquacote (Registered trademark), hydroxyethyl cellulose (Natrosol (Registered trademark), carboxymethyl cellulose, hydroxypropylmethyl cellulose, microcrystalline cellulose, hydroxybutylmethyl cellulose, hydroxypropyl cellulose (Klucel (Registered trademark), hydroxyethyl ethyl cellulose, cetyl hydroxyethyl cellulose (Natrosol (Registered trademark Plus 330), N-vinylpyrollidone (Povidone (Registered trademark), Acrylates/Ceteth-20 Itaconate Copolymer (Structure (Registered trademark 3001), hydroxypropyl starch phosphate (Structure (Registered trademark ZEA), polyethoxylated urethanes or polycarbamyl polyglycol ester (e.g. PEG-150/Decyl/SMDI copolymer=Aculyn (Registered trademark 44, PEG-150/Stearyl/SMDI copolymer=Aculyn 46 (Registered trademark), trihydroxystearin (Thixcin (Registered trademark) acrylates copolymer (e.g. Aculyn (Registered trademark 33) or hydrophobically modified acrylate copolymers (e.g. Acrylates/Steareth-20 Methacrylate Copolymer=Aculyn (Registered trademark 22), and fatty alcohols, such as cetyl and stearyl alcohol, and combinations thereof.

### Conditioner Composition

The personal care composition of the present invention can be a hair conditioner. The hair conditioner composition delivers consumer desired benefits such as wet feel, combability, color retention, protection against hair damage, damage repair, dry feel, anti-frizz benefits, etc. shampooing in addition to scalp anti-dandruff efficacy benefit.

The conditioner composition may comprise rinse off conditioners. In addition, it may comprise other optional ingredients such as silicone or organic conditioning agents, hair health actives, anti-dandruff actives, and other ingredients.

Hair conditioners are typically applied on hair after rinsing the shampoo composition from the hair. The conditioner composition described herein delivers consumer desired hair conditioning in addition to anti-dandruff benefits.

The conditioner composition described herein may also comprise a conditioner gel matrix comprising (1) one or more high melting point fatty compounds, (2) a cationic surfactant system, and (3) a second aqueous carrier. After applying to the hair a conditioner composition, the conditioner is rinsed from the hair using water.

A. Cationic Surfactant System

The conditioner gel matrix of the conditioner composition includes a cationic surfactant system. The cationic surfactant system can be one cationic surfactant or a mixture of two or more cationic surfactants. The cationic surfactant system can be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt.

The cationic surfactant system can be included in the composition at a level by weight of from about 0.1% to about 10%, from about 0.5% to about 8%, from about 0.8% to about 5%, and from about 1.0% to about 4%.

### Mono-Long Alkyl Quaternized Ammonium Salt

The monoalkyl quaternized ammonium salt cationic surfactants useful herein are those having one long alkyl chain which has about 22 carbon atoms and in may be a C22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

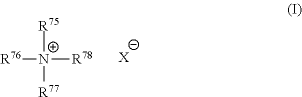

wherein one of R⁷⁵, R⁷⁶, R⁷⁷ and R⁷⁸ is selected from an alkyl group of 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of about 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of about 22 carbons. Exemplary tertiary amido amines include: behenamidopropyldimethylamine, behenamidopropyldiethylamine, behenamidoethyldiethylamine, behenamidoethyldimethylamin. Useful amines in the present invention are disclosed in U.S. Pat. No. 4,275,055, Nachtigal, et al. These amines can also be used in combination with acids such as □-glutamic acid, lactic acid, hydrochloric acid, malic acid, succinic acid, acetic acid, fumaric acid, tartaric acid, citric acid, □-glutamic hydrochloride, maleic acid, and mixtures thereof; and may be □-glutamic acid, lactic acid, and/or citric acid. The amines herein can be partially neutralized with any of the acids at a molar ratio of the amine to the acid of from about 1:0.3 to about 1:2, and/or from about 1:0.4 to about 1:1.

### Di-Long Alkyl Quaternized Ammonium Salt

Di-long alkyl quaternized ammonium salt can be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of from about 10% to about 50%, and/or from about 30% to about 45%.

The di-long alkyl quaternized ammonium salt cationic surfactants useful herein are those having two long alkyl chains having about 22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

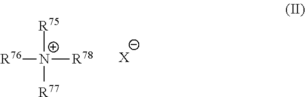

wherein two of R⁷⁵, R⁷⁶, R⁷⁷ and R⁷⁸ is selected from an alkyl group of from 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of from 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (C22) dimethyl ammonium chloride, ditallow alkyl dimethyl ammonium chloride, dihydrogenated tallow alkyl dimethyl ammonium chloride. Such dialkyl quaternized ammonium salt cationic surfactants also include, for example, asymmetric dialkyl quaternized ammonium salt cationic surfactants.

B. High Melting Point Fatty Compound

The conditioner gel matrix of the conditioner composition includes one or more high melting point fatty compounds. The high melting point fatty compounds useful herein may have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are suitable for use in the conditioner composition. The fatty alcohols useful herein are those having from about 14 to about 30 carbon atoms, from about 16 to about 22 carbon atoms. These fatty alcohols are saturated and can be straight or branched chain alcohols. Suitable fatty alcohols include, for example, cetyl alcohol, stearyl alcohol, behenyl alcohol, and mixtures thereof.

High melting point fatty compounds of a single compound of high purity can be used. Single compounds of pure fatty alcohols selected from the group of pure cetyl alcohol, stearyl alcohol, and behenyl alcohol can also be used. By “pure” herein, what is meant is that the compound has a purity of at least about 90%, and/or at least about 95%. These single compounds of high purity provide good rinsability from the hair when the consumer rinses off the composition.

The high melting point fatty compound can be included in the conditioner composition at a level of from about 0.1% to about 20%, alternatively from about 1% to about 15%, and alternatively from about 1.5% to about 8% by weight of the composition, in view of providing improved conditioning benefits such as slippery feel during the application to wet hair, softness and moisturized feel on dry hair.

### Leave-on Treatment

The personal care composition of the present invention can be a leave-on treatment. The leave-on treatment composition delivers consumer desired hair conditioning or styling benefit in addition to scalp anti-dandruff efficacy benefit.

The leave-on treatment composition may comprise dry shampoos, mousses, pastes, gels, and milks. The leave-on treatment may also comprise (1) one or more rheology modifiers. In addition, it may comprise pother optional ingredients such as silicone or organic conditioning agents, thickeners, hair health actives, anti-dandruff actives, and other ingredients.

Accordingly, the formulations of the leave-on treatment can be in the form of pourable liquids (under ambient conditions).

In cases where the leave-on composition does not include a gel matrix, it is preferred that the composition is pre-emulsified before added in the personal care composition. In cases where the leave-on composition does not include a gel matrix, it is preferred that the composition also comprises a rheology modifier/thickener.

In the present invention, the leave-on treatment may involve the application of a 1% w/w solution of the materials in a mixture of water, emulsifier and a thickener (Sepigel 305). Preferred materials include 1,2-decanediol, 1,2-dodecanediol, 1,2-octanediol for 1-2-diols and silica silylate, salicylic acid, 2,4-dihydroxy benzoic acid, 4-chlororesorcinol, 1,2,4-Trihydroxybenzene and zinc carbonate for solid particles.

The azoxystrobin containing product may be a liquid, solid or powder or combinations thereof and can be dispensed from a container or can be a single use product. Non-limiting examples of single use products may include a discrete product that is in the form of a solid foam, capsule, pill, pod, sheet, film, tablet, compressed powder, encapsulated liquid, pouch or fibers. A powder may be dispensed from a container or delivered from an aerosol as a dry shampoo. The product may also be a liquid cleansing composition that is rinsed off including for cleansing skin or hair including shampoo, conditioners, body wash, or facial cleansing. The personal care product may be a deodorant in the form of a solid or an aerosol or pump spray.

### pH

The personal care compositions mentioned above may also comprise one or more pH adjusting material. The compositions may have a pH in the range from about 2 to about 10, at 25° C. The rinse-off conditioner composition, and/or leave-on treatment may have a pH in the range of from about 2 to about 6, alternatively from about 3.5 to about 5, alternatively from about 5.25 to about 7.

The personal care compositions mentioned above may further comprise one or more pH buffering agent. Suitable buffering agents are well known in the art and include for example ammonia/ammonium acetate mixture and monoethanolamine (MEA). The rinse-off conditioner composition may comprise citric acid, wherein the citric acid acts as a buffer.

### Optional Ingredients

The conditioner compositions, pre-wash compositions and/or leave-on treatments described herein may optionally comprise one or more additional components known for use in personal care or personal care products, provided that the additional components are physically and chemically compatible with the essential components described herein, or do not otherwise unduly impair product stability, aesthetics or performance. Such additional components are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt. % to about 10 wt. % by weight of the personal care compositions.

Non-limiting examples of additional components for use in the personal care compositions include conditioning agents, natural cationic deposition polymers, synthetic cationic deposition polymers, other anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, proteins, skin active agents, sunscreens, UV absorbers, and vitamins.

1. Conditioning Agent

The personal care compositions may comprise one or more conditioning agents. Conditioning agents include materials that are used to give a particular conditioning benefit to hair. The conditioning agents useful in the personal care compositions of the present invention typically comprise a water-insoluble, water-dispersible, non-volatile, liquid that forms emulsified, liquid particles. Suitable conditioning agents for use in the personal care composition are those conditioning agents characterized generally as silicones, organic conditioning oils or combinations thereof, or those conditioning agents which otherwise form liquid, dispersed particles in the aqueous surfactant matrix.

One or more conditioning agents are present from about 0.01 wt. % to about 10 wt. %, from about 0.1 wt. % to about 8 wt. %, and from about 0.2 wt. % to about 4 wt. %, by weight of the composition.

### Silicone Conditioning Agent

The compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, epoxy groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the polymer can be in an emulsified form with droplet size of about 10 nm to about 30 micrometers

### Organic Conditioning Materials

The conditioning agent of the compositions of the present invention may also comprise at least one organic conditioning material such as oil or wax, either alone or in combination with other conditioning agents, such as the silicones described above. The organic material can be nonpolymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-20 200, PEG-400, PEG-600, PEG-1000, PEG-2M, PEG-7M, PEG-14M, PEG-45M and mixtures thereof.

### Benefit Agents

The personal care composition may further comprise one or more additional benefit agents. The benefit agents comprise a material selected from the group consisting of anti-dandruff agents, antifungal agents, anti-itch agents, anti-bacterial agents, anti-microbial agents, moisturization agents, anti-oxidants, vitamins, lipid soluble vitamins, perfumes, brighteners, enzymes, sensates, attractants, dyes, pigments, bleaches, and mixtures thereof.

The personal care compositions of the present invention may be presented in typical personal care formulations. They may be in the form of solutions, dispersion, emulsions, powders, talcs, encapsulated, spheres, spongers, solid dosage forms, foams, and other delivery mechanisms. The compositions of the present invention may be hair tonics, leave-on hair products such as treatment, and styling products, rinse-off hair products such as hair conditioners, and treatment products; and any other form that may be applied to hair. The personal care composition may be a hair mask, cowash, hair wax, hair clay, hair food, hair milk, hair pudding and hair gels.

The personal care compositions may be provided in the form of a porous, dissolvable solid structure, such as those disclosed in U.S. Patent Application Publication Nos. 2009/0232873; and 2010/0179083, which are incorporated herein by reference in their entirety. Accordingly, the personal care compositions comprise a chelant, a buffer system comprising an organic acid, from about 23% to about 75% surfactant; from about 10% to about 50% water soluble polymer; and optionally, from about 1% to about 15% plasticizer; such that the personal care composition is in the form of a flexible porous dissolvable solid structure, wherein said structure has a Percent open cell content of from about 80% to about 100%.

The personal care compositions may be in the form of a porous dissolvable solid structure comprising a chelant; a buffer system comprising an organic acid from about 23% to about 75% surfactant; wherein said surfactant has an average ethoxylate/alkyl ratio of from about 0.001 to about 0.45; from about 10% to about 50% water soluble polymer; and from about 1% to about 15% plasticizer; and wherein said article has a density of from about 0.03 g/cm³ to about 0.20 g/cm³.

The personal care compositions may be in the form of a viscous liquid comprising a chelant; a buffer system comprising an organic acid from 5-20% surfactant and a polycarboxylate rheology modifier; wherein the polycarboxylate is specifically chosen to be effective at the high electrolyte levels resulting from the incorporation of the key buffer system and chelant used for this invention. Non-limiting examples include acrylates/C10-C30 alkyl acrylate crosspolymers such as Carbopol EDT2020, 1342, 1382, etc. from Lubrizol. Rheology benefits of these actives may include stability, ease of dispensing, smoothness of spreading, etc.

The personal care compositions are generally prepared by conventional methods such as are known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The personal care composition may be in a single phase or a single product, or the personal care composition may be in a separate phases or separate products. If two products are used, the products may be used together, at the same time or sequentially. Sequential use may occur in a short period of time, such as immediately after the use of one product, or it may occur over a period of hours or days.

### Methods

### In Vitro Evaluation of Antifungal Potency

Malassezia furfur (CBS 7982) is grown for approximately 24 hours in a 250 ml vent cap polycarbonate Erlenmeyer flask containing approximately 100 ml of mDixon medium and 5 ml of a fully grown M. furfur culture prepared using the same conditions as described. The cells are diluted by mixing 2.5 ml of the 24 hour-old culture per 50 ml of mDixon medium. A Versette robot (ThermoFisher Scientific) is used to transfer 292.5 μl of the dilute cells into each well of a Beckman 267007 polypropylene round bottom deep well plate. All compounds are sourced from Sigma Aldrich (St. Louis, Mo.) and prepared as 10 mg/ml in DMSO. A semipermeable aeraseal membrane is applied to the plate which is then be covered with water-soaked cotton batting. The samples are be shaken at 31° C. on a Heidolph Titramax 100 shaker at 1500 rpm for approximately 72 hours. The plates are shaken at 1250 rpm on an Eppendorf MixMate shaker to disperse the cells before transferring 200 μl to a Corning 3596 polystyrene plate. The samples are shaken again at 950 rpm on a MixMate shaker before the absorbance at 600 nm is read using a Molecular Devices SpectraMax plate reader.

### In Vivo Evaluation of Scalp Health Efficacy

Subjects are enrolled in a double-blind, randomized study for all test groups and have Baseline and Week 3 measurements of scalp health taken for flaking, itch and scalp health endpoints. Subjects take home a test product(s) and are instructed on use of test products throughout the study. Test products are solubilized in a hydroalcoholic leave-on formulation. Azoxystrobin raw material is sourced from AK Scientific Inc. (Union City, Calif.) as >97% pure. Azoxystrobin scalp treatment is prepared from a slurry of azoxystrobin in ethanol that is formulated to 0.1% (w/v) in a base chassis of 50% ethanol, 0.35% Ultrez 21 (rheology modifier), 0.05% neutrol TE and water (q.s.). Vehicle scalp treatment is prepared similarly without the addition of azoxystrobin raw material. Subjects apply either azoxystrobin or vehicle scalp treatments daily for 3 weeks.

Grading of the scalp is performed by qualified expert graders for adherent scalp flaking scores (ASFS) as described in published methods. Detailed methods can be found in Journal of Dermatological Treatment 2014, 25, 232-236, incorporated by reference herein. Briefly, flaking severity of a subject is assessed by examination of the scalp which is illuminated by lighting that mimics daylight conditions. The scalp is divided into eight sections and each section is assessed for the presence of dandruff flakes that are adhering to the scalp skin using a 0 to 10 (increment of 2 units) scale. Loose flakes in the hair are not considered in the grading. The final, or total, ASFS is the sum of the grades for all eight scalp sections, which results in a scale ranging from 0 to 80 units. The change in flaking across time is reported as change from baseline at the 3-week time point.

Biomarkers of scalp health, including biomarkers of inflammation, itch, oxidative stress and barrier integrity are assessed by non-invasive tape strip sampling of the scalp surface to determine therapeutic resolution. All biomarkers are analyzed from D-Squame® scalp tape samples that are collected from the scalp of subjects before and after scalp treatment with either azoxystrobin or vehicle (Baseline, and Week 3 respectively). These are extracted and analyzed according to published methods (International Journal of Dermatology 2011, 50, 102-113, incorporated by reference herein). Data analysis is conducted by standard statistical methods and calculations. Briefly, D-Squame® tape strip samples (standard sampling discs, 22-mm diameter; CuDerm Corp., Dallas, Tex., USA tape strips are collected from each subject at each time-point (baseline and after 3 weeks of treatment) from the highest flaking octant as determined by the qualified grader at the baseline flaking assessment. The tape samples are collected by isolating the scalp skin by making a good parting in the hair (using a comb and clips). The tape is then placed on the parting and rubbed repeatedly (15-20 strokes) with the blunt end of a forceps to ensure that good contact is achieved. The tape is removed and a subsequent tape is placed on the same location and the process repeated until six sequential tapes have been collected.

Human inflammatory cytokines are analyzed to evaluate skin irritation and inflammatory processes. D-Squame® tape strips sampled from human scalp are extracted with phosphate-buffered saline (PBS) containing an additional 0.25M NaCl and a commercially available protease inhibitor cocktail comprising a mixture of protease inhibitors with broad-spectrum inhibitory specificity (Roche Applied Science, Inc., Indianapolis, Ind., USA) for 30 min with sonication on ice. Multiple human cytokines (IL-1α, IL-IRA) are simultaneously quantified using a Milliplex Human Cytokine Multiplex Kit (Millipore Corp., Billerica, Mass., USA). Aliquots of these extracts are analyzed for soluble protein using the BCA™ Protein Assay Kit (Pierce Biotechnology/Thermo Scientific, Rockford, Ill., USA) using bovine serum albumin (BSA) as a reference standard. Cytokine data are reported as pg/μg soluble protein.

Histamine is analyzed by gradient reversed-phase HPLC/MS/MS to evaluate scalp itch. D-Squame® tape strips sampled from human scalp are placed into individual polypropylene vials, and each vial is spiked with a stable isotope-labeled histamine (D4-histamine) internal standard (ISTD) and then extracted with acidified water (0.1% formic acid in distilled-deionized water) using sonication for 10 min. Each extract solution is isolated from the tape strip and an aliquot of each sample is placed into a specified position of a 96-well polypropylene plate. A set of histamine standards are prepared in the 96-well polypropylene plate over an appropriate calibration range in the acidified water and spiked with ISTD. The standards and the extracts of the scalp tape strips are analysed using gradient reversed-phase HPLC/MS/MS. Histamine and the ISTD are monitored by positive ion electrospray (ESI) using multiple reaction monitoring with the precursor ions of 112 m/z (histamine) and 116 m/z (ISTD) and product ions 95 m/z (histamine) and 99 m/z (ISTD). A standard curve is constructed by plotting the signal, defined here as the peak area ratio (peak area histamine/peak area ISTD), for each standard vs. the mass of histamine for the corresponding standard. The mass of histamine in the calibration standards and human scalp extract samples is then back-calculated using the generated regression equation. The result is reported as the mass of histamine (ng)/μg of protein that is found in the tape strip extract as determined with the BCA™ Protein Assay Kit (Pierce Biotechnology/Thermo Scientific, Rockford, Ill., USA).

S100A12 protein is analyzed to determine the impact of scalp treatment on active inflammatory disease. D-Squame® tape strips sampled from human scalp are extracted with standard extraction buffers and extracts are analyzed with a custom antibody kit for S100A12 from Meso Scale Discovery (Rockville, Md.). The resulting amount of S100A12 is also standardized by dividing by the amount of soluble protein measured in the tape strip extract.

Myeloperoxidase protein is analyzed to determine the impact of scalp treatment oxidative stress and potential oxidative damage. D-Squame® tape strips sampled from human scalp are extracted with standard extraction buffers and extracts are analyzed with a myeloperoxidase immunoassay kit from Meso Scale Discovery (Rockville, Md.). The resulting amount of myeloperoxidase is also standardized by dividing by the amount of soluble protein measured in the tape strip extract.

Biochemical markers of skin/scalp barrier integrity are analyzed to evaluate improvements on barrier health. D-Squame® tape strip samples of human scalp skin are extracted with PBS containing 0.2% SUS and 0.5% propylene glycol (PG) for 30 min with sonication on ice. Human skin analytes (keratin 1, keratin 10, human serum albumin) are simultaneously quantified using a 3-plea Human Skin Panel LINCOplex Kit (Millipore Corp.). Soluble protein is measured using the BCA™ Protein Assay Kit. Barrier integrity data are reported as either pg/μg or ng/μg soluble protein.

Self-assessment questionnaires for itch perception are administered to each subject at Baseline and Week 3. Itch perception is quantified by each subject as severity of symptoms over the past 24 hours on a 7 point scale: 0=none, 1=slight, 2=slight to moderate, 3=moderate, 4=moderate to severe; 5=severe; 6=very severe.

### Results

### Antifungal Potency

The data below demonstrates that Azoxystrobin is unique among other strobilurins in its exceptionally strong antifungal potency against Malassezia yeasts. Azoxystrobin exhibits a minimal inhibitory concentration (MIC) of 0.39-0.49 ppm against Malassezia furfur which is 4 times more potent than pyraclostrobin (1.95 ppm), the next most potent strobilurin and 8 times more potent than zinc pyrithione (3.13 ppm), a commonly used antifungal-based anti-dandruff active. Orysastrobin, another strobilurin antifungal agent used in agriculture produced an MIC of 31.25 ppm which is 64 times less potent than azoxystrobin.

Malassezia furfur

| Test Compound | MIC value (ppm) |
|---|---|
| Azoxystrobin | 0.39-0.49 |
| Pyraclastrobin | 1.95 |
| Fluoxastrobin | 3.91 |
| Dimoxystrobin | 7.81 |
| Kresoxim-methyl | 15.63 |
| Trifloxystrobin | 15.63 |
| Orysastrobin | 31.25 |
| Zinc pyrithione | 3.13 |

### Scalp Flaking

The data below demonstrates the ability of azoxystrobin to reduce scalp flaking for scalp and hair benefits. The change from baseline in scalp flaking scores (ASFS) is analyzed using an analysis of covariance (ANCOVA) model. The model includes treatment, study site, gender, baseline measurement and age as covariates. Azoxystrobin reduces the adherent scalp flaking score (ASFS) by 4.7 units more than the placebo (with no azoxystrobin) (p=0.002) after 3 weeks of treatment. The dandruff clinical population in this example that completes the study and are evaluated based on enrollment criteria and adherence to study procedures are 53% female and 47% male. The overall flaking results reflect the potency of the treatment response with effectiveness that is demonstrated for both male and female subjects.

| | Flaking Results | P-Value |
|---|---|---|
| Scalp Treatment | (ASFS Change from Baseline) | (treatment comparison) |
| Azoxystrobin | −13.88 | 0.002 |
| Vehicle | −9.16 | — |

### Inflammation

The data below demonstrates that azoxystrobin significantly reduces pro-inflammatory biomarkers of scalp health in consumers after 3 weeks of treatment. The ratio of the interleukin-1 receptor antagonist to interleukin-1 (IL-1RA: IL-1α) which is established as elevated in dandruff as an indicator of an unhealthy scalp, is reduced by treatment with azoxystrobin. Azoxystrobin scalp treatment also significantly reduces the levels of S100A12, a cytokine-like antimicrobial peptide that is expressed in inflammatory disease compared to placebo control (p=0.1042).

| Scalp Treatment | % Change from Baseline |
|---|---|
| Azoxystrobin | −58.3* |
| Vehicle | −27.7 |

*Indicates a significant difference between treatments (P <= 0.20).

| | S100A12 Results |
|---|---|
| Scalp Treatment | % Change from Baseline |
| Azoxystrobin | −71.2* |
| Vehicle | −55.4 |

*Indicates a significant difference between treatments (P <= 0.20).

### Itch

The data below shows that azoxystrobin does not significantly reduce histamine compared to placebo control or baseline values. Nevertheless, subjects in the azoxystrobin treatment group perceive a reduction in itch on the scalp that is significantly improved from itch severity at baseline and itch severity perceived by subjects in the placebo control treatment group. This indicates that Azoxystrobin is surprisingly able to provide an itch benefit although it does not function to reduce itch via reduction of histamine and the histamine-dependent pathway of scalp itch in dandruff sufferers that has been previously established as directly proportional to a reduction in itch perception.

| | Histamine Results |
|---|---|
| Scalp Treatment | % Change from Baseline |
| Azoxystrobin | 23.8 |
| Vehicle | 9.1 |

| | Mean Itch Severity at Week 3 |
|---|---|
| Scalp Treatment | Common Baseline Severity = 3.1 |
| Azoxystrobin | 1.5* |
| Vehicle | 2.0 |

*p-value = 0.05 for treatment mean pairwise comparison between azoxystrobin and vehicle

### Oxidative Stress

The data below demonstrates that azoxystrobin significantly reduces myeloperoxidase, a biochemical marker of oxidative stress after 3 weeks of treatment. Myeloperoxidase is an enzyme that is elevated as part of the immune response to defend against microbial pathogens. Its chemical defense against pathogens, including Malassezia involves production of hypochlorous acid, a powerful oxidant that serves a biocidal function but also produces toxic reactive oxygen species which can result in collateral tissue damage. Oxidative stress and resulting damage of scalp tissue has established impact to both scalp health and hair health (International Journal of Trichology 2018, 10, 262-270, incorporated by reference herein. Azoxystrobin reduction of scalp myeloperoxidase indicates benefits for reduced oxidative damage for scalp which has consequences for scalp and hair health.

| | Myeloperoxidase Results |
|---|---|
| Scalp Treatment | % Change from Baseline |
| Azoxystrobin | 63.7* |
| Vehicle | 13.7 |

*Indicates a significant difference between treatments (P <= 0.20).

### Epidermal Barrier Health

Azoxystrobin scalp treatment significantly modulates biochemical markers of epidermal barrier integrity as indicators of scalp health benefits. An intact epidermal barrier is the first line of defense against bacteria, fungi, pollution and other environmental insults. Azoxystrobin significantly reduces human serum albumin (HSA) which indicates that the scalp barrier becomes less leaky and permeable to external insults. Azoxystrobin also significantly increases keratins 1 and 10 relative to placebo control which is indicative of improved epidermal barrier composition and function.

% Change from Baseline

| Treatment | Human Serum Albumin | Keratins-1,10 |
|---|---|---|
| Azoxystrobin | −54.1* | +86.0* |
| Vehicle | −18.6 | +44.2 |

*Indicates a significant difference between treatments (P <= 0.20).

The present invention is directed to use of azoxystrobin to decrease the number of flakes on a surface. The present invention is directed to use of azoxystrobin to decrease itch on a surface as perceived by a user. The present invention is directed to use of azoxystrobin to decrease the level of myeloperoxidase on a surface as an indicator of decrease of oxidative stress and damage. The present invention is directed to use of azoxystrobin wherein the surface is selected from the group consisting of skin, scalp, or human scalp and mixtures thereof.

The present invention is directed to use of azoxystrobin wherein there is a reduction in an adherent scalp flaking score (ASFS) by 4.7 units with a 45% reduction in flaking compared to compared to a placebo control and a 30% reduction in flaking after 3 weeks of treatment. The present invention is directed to use of azoxystrobin wherein there is a 58% reduction of Interleukin-1 (IL-1RA: IL-1α) as compared to a placebo control. The present invention is directed to use of azoxystrobin wherein there is a 71% reduction of S100A12 levels as compared to a placebo control. The present invention is directed to use of azoxystrobin wherein there is an improvement in scalp health barrier by a 54% reduction in human serum albumin (HSA) changed from a baseline.

### Examples

### Non-limiting Examples

The shampoo compositions illustrated in the following examples are prepared by conventional formulation and mixing methods. All exemplified amounts are listed as weight percents on an active basis and exclude minor materials such as diluents, preservatives, color percentages are based on weight unless otherwise specified.

### Shampoo Examples

| Note | Component/Example | 1 | 2 | 3 | 4 |
|---|---|---|---|---|---|
| | Water | 83.09 | 83.4 | 86.6 | 86.98 |
| 1 | Sodium laureth-1 sulfate | 11 | 14 | 0 | 6 |
| 2 | Cocamidopropyl betaine | 1 | 0 | 0 | 7 |
| 3 | CMEA | 1 | 2 | 0 | 0 |
| 4 | Sodium lauroyl sarcosinate | 0 | 0 | 5 | 0 |
| 5 | Decyl glucoside | 0 | 0 | 5 | 0 |
| 6 | Sodium laureth sulfosuccinate | 0 | 0 | 3 | 0 |
| 7 | Hydroxypropyl Methylcellulose | 0.3 | 0.1 | 0.2 | 0 |
| 8 | 1,10-Decanediol | 0 | 0 | 0.1 | 0 |
| | Azoxystrobin | 0.8 | 0.5 | 0.1 | 0.02 |
| 9 | Ethylene glycol distearate | 1.8 | 0 | 0 | 0 |
| 10 | Dimethiconol | 1 | 0 | 0 | 0 |

All above are on active basis; e.g. 11% SLE1S would require an addition of 44% of a 25% active SLE1S solution. The below table explains each Note from the above table

| | |
|---|---|
| 1 | Supplied at 25% active by Stepan |
| 2 | Supplied at 30% active by Evonik |
| 3 | Supplied at 85% active by BASF |
| 4 | Supplied at 30% active by Croda |
| 5 | Supplied as 50% active by BASF |
| 6 | Supplied as 35% active by Solvay |
| 7 | Supplied by Dow |
| 8 | Supplied by Symrise |
| 9 | Supplied by Evonik |
| 10 | Supplied by Wacker |

The following examples further describe and demonstrate unlimiting examples within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

Conditioner Compositions (wt %)

| Components | Ex. 5 | Ex 6 | Ex. 7 | Ex. 8 | Ex. 9 | Ex. 10 |
|---|---|---|---|---|---|---|
| Behenyl | 3.42 | 3.42 | | | 2.85 | 3.42 |
| trimethylammonium | | | | | | |
| Chloride/IPA*1 | | | | | | |
| Stearylamidopropyl | | | 2.40 | 2.40 | | |
| dimethylamine | | | | | | |
| Polysorbate 20 | 0.03 | | | | | 0.03 |
| L-glutamic acid | | | 0.77 | 0.77 | | |
| Citric acid | 0.22 | 0.10 | 0.20 | 0.30 | 0.06 | 0.22 |
| Cetyl alcohol | 1.67 | 1.67 | 2.50 | 2.50 | 1.67 | 1.67 |
| Stearyl alcohol | 4.18 | 4.18 | 4.50 | 4.50 | 4.18 | 4.18 |
| Azoxystrobin | 0.25 | 0.016 | 0.05 | 1.00 | 0.30 | 0.32 |
| Benzyl alcohol | 0.4 | 0.4 | 0.4 | 0.4 | 0.4 | 0.4 |
| Phenoxy ethanol | 0.4 | 0.4 | 0.4 | 0.4 | 0.4 | 0.4 |
| Sodium benzoate | 0.25 | 0.25 | 0.25 | 0.25 | 0.25 | 0.25 |
| Polydimethylsiloxane | 3.00 | | | 0.85 | 1.40 | |
| Deionized Water | q.s. to 100% | | | | | |
| pH | 4.1 | 4.9 | 5.0 | 4.2 | 5.9 | 5.9 |

### LEAVE-ON TREATMENT EXAMPLES

| | Component/ | | | | | | | | | | |
|---|---|---|---|---|---|---|---|---|---|---|---|
| Note | Example | 11 | 12 | 13 | 14 | 15 | 16 | 17 | 18 | 19 | 20 |
| | Water | 62.85 | 56.2 | 40.7 | 64.73 | 41.0 | 92.45 | 95.66 | 95.2 | 92.7 | 92.5 |
| 1 | Ethanol | 30 | 40 | 50 | 30 | 50 | 0 | 0 | 0 | 0 | 0 |
| 2 | 2-Pyrrolidinone, | 3.5 | 0 | 4 | 0 | 0 | 2 | 0 | 2 | 0 | 0 |
| | 1-ethenyl-, | | | | | | | | | | |
| | homopolymer | | | | | | | | | | |
| | Azoxystrobin | 0.2 | 0.5 | 2 | 0.02 | 0.6 | 0.4 | 0.04 | 0.1 | 0.25 | 0.3 |
| 3 | Hydroxypropyl | 0 | 0 | 0 | 2 | 0 | 0 | 0 | 0 | 2 | 0 |
| | Methylcellulose | | | | | | | | | | |
| 4 | Hydroxypropyl | 0 | 0 | 0 | 0 | 5 | 0 | 0 | 0 | 0 | 3 |
| | Starch Phosphate | | | | | | | | | | |
| 5 | Menthol | 0.25 | 0.3 | 0.3 | 0.25 | 0.4 | 0.25 | 0.3 | 0.3 | 0.25 | 0.4 |
| 6 | Niacinamide | 2.5 | 2.5 | 2.5 | 2.5 | 2.5 | 2.5 | 2.5 | 0 | 2.5 | 2.5 |
| 7 | Caffeine | 0.1 | 0.1 | 0.1 | 0.1 | 0.1 | 0.1 | 0.1 | 0.1 | 0 | 0.1 |
| 8 | Panthenol | 0.1 | 0.1 | 0.1 | 0.1 | 0.1 | 0.1 | 0.1 | 0.1 | 0.1 | 0 |
| 9 | PEG-40 | 0 | 0 | 0 | 0 | 0 | 1 | 1 | 1 | 1 | 1 |
| | Hydrogenated | | | | | | | | | | |
| | Castor Oil | | | | | | | | | | |
| 10 | Propylene Glycol | 0 | 0 | 0 | 0 | 0 | 0.9 | 0 | 0.9 | 0.9 | 0 |
| | Fragrance | 0.5 | 0.3 | 0.3 | 0.3 | 0.3 | 0.3 | 0.3 | 0.3 | 0.3 | 0.2 |

1 SD-40B 200 Alcohol from Pride Solvents
2 Flexithix from Ashland
3 Benecel K200M from Ashland
4 Structure XL from AkzoNobel
5 Menthol from Kerry Ingredients and Flavors
6 Niacinamide from Lonza
7 Caffeine from Merck
8 D-Panthenol from BASF
9 Cremophor RH-40 from BASF
10 Propylene Glycol from Sigma Aldrich

### Combinations

Paragraph A Use of azoxystrobin to decrease the number of flakes on a surface.
Paragraph B Use of azoxystrobin according to Paragraph A to decrease itch on a surface as perceived by a user.
Paragraph C Use of azoxystrobin according to Paragraph A-B to decrease the level of myeloperoxidase on a surface as an indicator of decrease of oxidative stress and damage.
Paragraph D Use of azoxystrobin according to Paragraph A-C wherein the surface is selected from the group consisting of skin, scalp, or human scalp and mixtures thereof.
Paragraph E Use of azoxy according to Paragraph A-D wherein the surface is selected from the group consisting of skin, scalp or human scalp and mixtures thereof.
Paragraph F Use of azoxystrobin according to Paragraph A-E wherein the surface is selected from the group consisting of skin, scalp or human scalp and mixtures thereof.
Paragraph G Use or azoxystrobin according to Paragraph A-F wherein the level of azoxystrobin is from about 0.01% to about 10%.
Paragraph H Use or azoxystrobin according to Paragraph A-G wherein the level of azoxystrobin is from about 0.01% to about 10%.
Paragraph I Use or azoxystrobin according to Paragraph A-H wherein the level of azoxystrobin is from about 0.01% to about 10%.
Paragraph J Use of azoxystrobin according to Paragraph A-I, wherein the azoxystrobin is applied from a personal care composition.
Paragraph K Use of azoxystrobin according to Paragraph A-J, wherein the azoxystrobin is applied from a personal care composition.
Paragraph L Use of azoxystrobin according to Paragraph A-K, wherein the azoxystrobin is applied from a personal care composition.
Paragraph M Use of azoxystrobin according to Paragraph A-L wherein the personal care composition is selected from the group consisting of a leave on treatment, a shampoo, or a conditioner.
Paragraph N Use of the azoxystrobin according to Paragraph A-M wherein the personal care composition is selected from the group consisting of a leave on treatment, a shampoo, or a conditioner.
Paragraph O Use of azoxystrobin according to Paragraph A-N wherein the personal care composition is selected from the group consisting of a leave on treatment, a shampoo, or a conditioner.
Paragraph P Use of azoxystrobin according to Paragraph A-O wherein the personal care composition is selected from the group consisting of a leave on treatment, a shampoo, or a conditioner.
Paragraph Q Use of azoxystrobin according to Paragraph A-P wherein the personal care composition is selected from the group consisting of a leave on treatment, a shampoo, or a conditioner.
Paragraph R Use of azoxystrobin according to Paragraph A-Q wherein the personal care composition is selected from the group consisting of a leave on treatment, a shampoo, or a conditioner.
Paragraph S Use of azoxystrobin according to Paragraph A-R wherein there is a reduction in an adherent scalp flaking score (ASFS) by 4.7 units with a 45% reduction in flaking compared to compared to a placebo control and a 30% reduction in flaking after 3 weeks of treatment.
Paragraph T Use of azoxystrobin according to Paragraph A-S, wherein there is a 58% reduction of Interleukin-1 (IL-1RA: IL-1a) as compared to a placebo control.
Paragraph U Use of azoxystrobin according to Paragraph A-T wherein there is a 71% reduction of S100A12 levels as compared to a placebo control.
Paragraph V Use of azoxystrobin according to Paragraph A-U wherein there is an improvement in scalp health barrier by a 54% reduction in human serum albumin (HSA) changed from a baseline.
Paragraph W Use of azoxystrobin according to Paragraph A-V wherein there is a 86% increase in keratins 1 and 10 as compared to a placebo control.
Paragraph X. Use of azoxystrobin according to Paragraph A-W wherein there is a 48% reduction in an itch perception as compared to a placebo control.
Paragraph Y Use of azoxystrobin according to Paragraph A-X wherein there is a 64% reduction in myeloperoxidase as compared to a placebo control.
Paragraph Z Use of azoxystrobin according to Paragraph A-Y wherein the personal care composition further comprises from 2% to 50% of one or more anionic surfactant.
Paragraph AA Use of azoxystrobin according to Paragraph A-Z wherein the personal care composition further comprises from 2% to 50% of one or more anionic surfactant.
Paragraph BB Use of azoxystrobin according to Paragraph A-AA wherein the personal care composition further comprises from 2% to 50% of one or more anionic surfactant.
Paragraph CC Use of azoxystrobin according to paragraph A-BB wherein the personal care composition further comprises a nonionic, amphoteric, cationic or zwitterionic surfactant and mixtures thereof.
Paragraph DD Use of azoxystrobin according to Paragraph A-CC wherein the personal care composition further comprises a nonionic, amphoteric, cationic or zwitterionic surfactant and mixtures thereof.
Paragraph EE Use of azoxystrobin according to Paragraph A-CD wherein the personal care composition further comprises a nonionic, amphoteric, cationic or zwitterionic surfactant and mixtures thereof.
Paragraph FF Use of azoxystrobin according to Paragraph A-EE wherein the personal care composition further comprises a cationic polymer.
Paragraph GG Use of azoxystrobin according to Paragraph A-FF wherein the personal care composition further comprises a cationic polymer.
Paragraph HH Use of azoxystrobin according to Paragraph A-GG wherein the personal care composition further comprises a cationic polymer.
Paragraph II Use of azoxystrobin according to Paragraph A-HH wherein the personal care composition further comprises a conditioning agent.
Paragraph JJ Use of azoxystrobin according to Paragraph A-II wherein the personal care composition further comprises a conditioning agent.
Paragraph KK Use of azoxystrobin according to Paragraph A-JJ wherein the personal care composition further comprising a conditioning agent.

Product Forms

The personal care compositions of the present invention may be presented in typical personal care formulations. They may be in the form of solutions, dispersion, emulsions, powders, talcs, encapsulated, spheres, spongers, solid dosage forms, foams, and other delivery mechanisms. The compositions of the present invention may be hair tonics, leave-on hair products such as treatment, and styling products, rinse-off hair products such as shampoos, pre-wash product, co-wash product, and personal cleansing products, and treatment products; and any other form that may be applied to hair or skin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature.

All documents cited in the Detailed Description of the Invention are, in relevant part, incorporated herein by reference; the citation of any document is not to be construed as an admission that it is prior art with respect to the invention. To the extent that any meaning or definition of a term in this document conflicts with any meaning or definition of the same term in a document incorporated by reference, the meaning or definition assigned to that term in this document shall govern.

While particular embodiments of the invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. Use of azoxystrobin to decrease the number of flakes on a surface.

2. Use of azoxystrobin to decrease itch on a surface as perceived by a user.

3. Use of azoxystrobin to decrease the level of myeloperoxidase on a surface as an indicator of decrease of oxidative stress and damage.

4. Use of azoxystrobin according to claim 1 wherein the surface is selected from the group consisting of skin, scalp, or human scalp and mixtures thereof.

5. Use of azoxystrobin according to claim 2 wherein the surface is selected from the group consisting of skin, scalp or human scalp and mixtures thereof.

6. Use of azoxystrobin according to claim 3 wherein the surface is selected from the group consisting of skin, scalp or human scalp and mixtures thereof.

7. Use or azoxystrobin according to claim 1 wherein the level of azoxystrobin is from about 0.01% to about 10%.

8. Use or azoxystrobin according to claim 2 wherein the level of azoxystrobin is from about 0.01% to about 10%.

9. Use or azoxystrobin according to claim 3 wherein the level of azoxystrobin is from about 0.01% to about 10%.

10. Use of azoxystrobin according to claim 1, wherein the azoxystrobin is applied from a personal care composition.

11. Use of azoxystrobin according to claim 2, wherein the azoxystrobin is applied from a personal care composition.

12. Use of azoxystrobin according to claim 3, wherein the azoxystrobin is applied from a personal care composition.

13. Use of azoxystrobin according to claim 1, wherein the personal care composition is selected from the group consisting of a leave on treatment, a shampoo, a conditioner and mixtures thereof.

14. Use of azoxystrobin according to claim 2, wherein the personal care composition is a selected from the group consisting of a leave on treatment, a shampoo, a conditioner and mixtures thereof.

15. Use of azoxystrobin according to claim 3, wherein the personal care composition is selected from the group consisting of a leave on treatment, a shampoo, a conditioner and mixtures thereof.

16. Use of azoxystrobin according to claim 1 wherein there is a reduction in an adherent scalp flaking score (ASFS) by 4.7 units with a 45% reduction in flaking compared to compared to a placebo control and a 30% reduction in flaking after 3 weeks of treatment.

17. Use of azoxystrobin according to claim 3 wherein there is a 58% reduction of Interleukin-1 (IL-1RA: IL-1α) as compared to a placebo control.

18. Use of azoxystrobin according to claim 3 wherein there is a 71% reduction of S100A12 levels as compared to a placebo control.

19. Use of azoxystrobin according to claim 3 wherein there is an improvement in scalp health barrier by a 54% reduction in human serum albumin (HSA) changed from a baseline.

20. Use of azoxystrobin according to claim 3 wherein there is an 86% increase in keratins1 and 10 as compared to a placebo control.

21. Use of azoxystrobin according to claim 2 wherein there is a 48% reduction in an itch perception as compared to a placebo control.

22. Use of azoxystrobin according to claim 3 wherein there is a 64% reduction in myeloperoxidase as compared to a placebo control.

23. Use of azoxystrobin according to claim 1 wherein the personal care composition further comprises from 2% to 50% of one or more anionic surfactant.

24. Use of azoxystrobin according to claim 2 wherein the personal care composition further comprises from 2% to 50% of one or more anionic surfactant.

25. Use of azoxystrobin according to claim 3 wherein the personal care composition further comprises from 2% to 50% of one or more anionic surfactant.

26. Use of azoxystrobin according to claim 1 wherein the personal care composition further comprises a nonionic, amphoteric, cationic or zwitterionic surfactant and mixtures thereof.

27. Use of azoxystrobin according to claim 2 wherein the personal care composition further comprises a nonionic, amphoteric, cationic or zwitterionic surfactant and mixtures thereof.

28. Use of azoxystrobin according to claim 3 wherein the personal care composition further comprises a nonionic, amphoteric, cationic or zwitterionic surfactant and mixtures thereof.

29. Use of azoxystrobin according to claim 1 wherein the personal care composition further comprises a cationic polymer.

30. Use of azoxystrobin according to claim 2 wherein the personal care composition further comprises a cationic polymer.

31. Use of azoxystrobin according to claim 3 wherein the personal care composition further comprises a cationic polymer.

32. Use of azoxystrobin according to claim 1 wherein the personal care composition further comprises a conditioning agent.

33. Use of azoxystrobin according to claim 2 wherein the personal care composition further comprises a conditioning agent.

34. Use of azoxystrobin according to claim 3 wherein the personal care composition further comprising a conditioning agent.
